(19) United States
(12) Patent Application Publication
(10) Pub. No.: US 2011/0269237 A1
(43) Pub. Date: Nov. 3, 2011

(54) CATALYTICALLY-GENERATED GAS IN HYDROCARBON BEARING SOURCE ROCKS

(75) Inventor: Frank D. Mango, Houston, TX (US)

(73) Assignee: PETROLEUM HABITATS, L.L.C., Houston, TX (US)

(21) Appl. No.: 13/142,350

(22) PCT Filed: Dec. 29, 2009
(86) PCT No.: PCT/US2009/069753

§ 371 Date: Jun. 27, 2011

Related U.S. Application Data
(60) Provisional application No. 61/141,210, filed on Dec. 29, 2008.

(51) Int. Cl. G01N 33/24

(52) U.S. Cl. 436/32

(57) ABSTRACT

The present disclosure is directed to assaying rock samples (e.g., core samples) for the presence of catalytically-generated gases, such as methane for example. According to one or more aspects of the present disclosure, a method for assaying a rock sample comprises sealing a carbonaceous rock sample in a container having an atmosphere and assaying for a quantity of catalytically-generated gas in the sealed container. The method may comprise generating the catalytically-generated gas in response to a catalytic reaction between the carbonaceous material in the carbonaceous rock sample and a low-valent transition metal that is present in the carbonaceous rock sample. The catalytic reaction may occur in a static atmosphere of the sealed container.

### BACKGROUND

This section provides background information to facilitate a better understanding of the various aspects of the present invention. It should be understood that the statements in this section of this document are to be read in this light, and not as admissions of prior art.

Oil is known to progress to natural gas in deep sedimentary basins. This process, hereinafter referred to as “oil-to-gas,” is believed to be the major source of natural gas (primarily methane) in the earth. Knowing when and how this process occurs can provide a predictive model for oil and gas exploration.

A conventional view of oil-to-gas conversion is that oil thermally cracks to gas (thermal gas) at temperatures between 150° C. and 200° C. Temperatures in this range are commonly observed geologically where most oil-to-gas is observed. However, various kinetic models based on thermal gas have had only marginal predictive success in drilling operations. There is mounting scientific evidence suggesting that oil should not crack to gas, even over geologic time periods, at temperatures between 150° C. and 200° C., the range within which most so-called thermal gas is formed. For example, gas produced by industrial thermal cracking of hydrocarbons is typically severely depleted in methane and does not resemble the natural gas distributed in the earth.

There is continuing desire to identify sources of hydrocarbons as an energy source. There is a still further desire to identify sources of natural gasses, for example, and without limitation, ethane to hexane.

### SUMMARY

The present disclosure is directed to assaying rock samples (e.g., core samples) for the presence of catalytically-generated gases, such as methane for example. According to one or more aspects of the present disclosure, a method for assaying a rock sample comprises sealing a carbonaceous rock sample in a container having an atmosphere and assaying for a quantity of catalytically-generated gas in the sealed container. The method may comprise generating the catalytically-generated gas in response to a catalytic reaction between the carbonaceous material in the carbonaceous rock sample and a low-valent transition metal that is present in the carbonaceous rock sample. The catalytic reaction may occur in a static atmosphere of the sealed container.

The foregoing has outlined some of the features and technical advantages of the present invention in order that the detailed description of the invention that follows may be better understood. Additional features and advantages of the invention will be described hereinafter which form the subject of the claims of the invention.

### BRIEF DESCRIPTION OF THE DRAWINGS

The present disclosure is best understood from the following detailed description when read with the accompanying figures. It is emphasized that, in accordance with standard practice in the industry, various features are not drawn to scale. In fact, the dimensions of various features may be arbitrarily increased or reduced for clarity of discussion.

FIG. 1 is an illustrative log of total organic carbon and C1-05 hydrocarbon yield in Barnett Shale by depth as assayed according to one or more aspects of a method of the present disclosure.

FIG. 2 is a graphical distribution of total C1-05 hydrocarbons catalytically-generated from a Floyd Shale as assayed in accordance to one or more aspects of the present disclosure.

FIG. 3 is a schematic depicting a well intersecting a subterranean target formation in accordance with one or more aspects of the present disclosure.

FIG. 4 is a schematic diagram of an embodiment of a method according to one or more aspects of the present disclosure.

FIG. 5 is a schematic diagram of another embodiment of a method according to one or more aspects of the present disclosure.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

While most of the terms used herein will be recognized by those of ordinary skill in the art, the following non-exhaustive list of terms is provide below to aid in understanding the present disclosure.

“Gas” as used herein, refers to natural gas. “Gas” may be utilized in particular to refer to the C1-C5 hydrocarbons. Various example and embodiments of the present disclosure are described with reference to methane for purposes of brevity and convenience.

“Inert gas” as used herein, refers to non-reactive gases such as, for example, helium, argon and nitrogen.

“Sedimentary rock” as used herein, refers to, for example, rock formed by the accumulation and cementation of mineral grains transported by wind, water, or ice to the site of deposition or chemically precipitated at the depositional site. Sedimentary rocks comprise, for example, reservoir rocks, source rocks, and conduit rocks. “Reservoir rocks” as used herein refer to, for example, subterranean material that traps and sequesters migrating fluids (e.g., from a reservoir formation). “Source rocks” as used herein refer to, for example, rocks within which petroleum is generated and either expelled or retained. “Conduit rocks” as used herein refer to, for example, rocks through which petroleum migrates from its source to its final destination (e.g., reservoir rock). A “sedimentary basin” as used herein, refers to, for example, a large accumulation of sediment, as in, for example, sedimentary rock. “Outcrop rocks” as used herein refer to, for example, segments of bedrock exposed to the atmosphere.

“Target reservoir” as used herein, refers to, for example, a drilling prospect in a sedimentary basin or other geological formation containing sedimentary rocks and believed to contain petroleum (e.g., oil and/or gas).

“Gas habitat” as used herein, refers to, for example, sedimentary rock within a sedimentary basin that is sufficiently catalytic to convert 90% or more of its contained oil to gas over a specified time interval at a given temperature.

“Oil habitat” as used herein, refers to, for example, sedimentary rock within a sedimentary basin that is not sufficiently catalytic to convert 90% or more of its contained oil to gas over a specified time interval at a given temperature.

“Oil-to-gas” as used herein, refers to, for example, geological processes in which crude oil containing higher molecular weight hydrocarbons is converted into natural gas containing lower molecular weight hydrocarbons such as, for example, methane and other C2-C5 hydrocarbons. “Transition metal” as used herein, refers to, for example, metals residing within the “d-block” of the Periodic Table. Specifically, these include elements 21-29 (scandium through copper), 39-47 (yttrium through silver), 57-79 (lanthanum through gold), and all known or unknown elements from 89 (actinium) onward. Illustrative transition metals with relevance in catalytic oil-to-gas conversion include, for example, iron, cobalt and nickel.

“Low-valent transition metals (LVTMs)” as used herein refer to, for example, transition metals that are in a low oxidation state. A low oxidation state for LVTMs may include, for example, a 0, +1, +2 or +3 oxidation state. “Zero-valent transition metals (ZVTMs)” as used herein refer to, for example, transition metals in their zero-oxidation (i.e., neutral) state.

“Quantitative analysis” as used herein, refers to, for example, a determination of species quantity and/or concentration with a specified high level of precision. “Qualitative analysis” as used herein, refers to, for example, a determination of species quantity and/or concentration with a lower level of precision than a quantitative analysis. A qualitative analysis is still at a level of precision capable of being used for predictive determinations.

“Assay” as used herein, refers to, for example, a quantitative and/or qualitative analysis of hydrocarbon gasses catalytically-generated from a rock sample under experimental conditions. The hydrocarbon gas measured by the assay is catalytically-generated by natural catalysis over the course of experimental time as opposed to pre-existing gas in the rock sample generated over geologic time.

“Catalytically-generated gas (CGG)” as used herein, refers to, for example, catalytically-generated methane (CGM) generated via a catalytic decomposition of a carbonaceous material (e.g., a hydrocarbon) catalyzed by ZVTM or LVTM. Catalytically-generated gas may be formed either under geological or laboratory conditions.

“Intrinsic catalytic activity” refers to, for example, the catalytic activity for oil-to-gas conversion of a rock sample, without the rock sample being compromised by exposure to oxygen. Intrinsic catalytic activity correlates with the native catalytic activity of the rock sample in the source reservoir from which the rock sample was obtained. In some embodiments of the present disclosure, the intrinsic catalytic activity may correlate with the amount of gas capable of being catalytically-generated in the source reservoir.

“Genetically-similar reservoir” as used herein, refers to, for example, a reservoir that is similar in overall organic and inorganic composition to a source reservoir and both of which were deposited under similar geological environments. Rocks from genetically-similar reservoirs can be expected to contain similar concentrations of transition metals and possess similar levels of catalytic activity.

“Habitat maps” as used herein refer to, for example, maps of stratigraphic rock units showing the lines of intersection between oil and gas habitats.

According to one or more aspects of the present disclosure, methods for assaying a rock to determine the rock's capability for catalytically generating gas (e.g., methane and other lower hydrocarbons such as ethane through hexane) is provided. According to one or more aspects of the present disclosure, subterranean reservoirs for hydrocarbon exploration and sustained production of natural gas can be predicted on the ability of a reservoir rock's capability for catalytically generating gas. According to on or more aspects of the present disclosure, catalytic gas generation capabilities may be determined and/or predicted from outcrop rocks, drill cuttings and core samples. According to one or more aspects of the present disclosure, the assays may be performed all or in part in a laboratory setting or downhole in a well (e.g., wellbore).

Catalytic conversion of hydrocarbons into natural gas mediated by transition metals is an explanation for geologic formation of gas. For example, crude oils can be catalytically converted to gas over zero-valent transition metals (ZVTM) such as, for example, Ni, Co, and Fe under anoxic conditions at moderate temperatures (150-200° C.). The catalytically-formed gas is typically identical or substantially similar to geologically-formed gas.

According to one or more aspects of the present disclosure, catalytic conversion of hydrocarbons into gas is considered as a viable gas production mechanism in sedimentary basins and other geological structures. Exposure of existing ZVTMs or low-valent transition metals (LVTMs) within the sedimentary rocks may result in catalytic activity for oil-to-gas conversion. Likewise, reduction of existing higher valent transition metals into ZVTMs or LVTMs can result in catalytic activity for oil-to-gas conversion in petroleum habitats, which typically exist with reducing conditions. Thus, according to one or more aspects of the present disclosure, a subterranean reservoir that contains high-activity source rocks may produce gas from the following two sources (e.g., mechanisms): 1) pre-existing gas that was generated over the course of geologic time and 2) catalytically-generated gas that was created during production (e.g., via a well) from the reservoir. Therefore, assays of rock samples from a reservoir or indicative of the reservoir may indicate how much additional catalytically-generated gas will be generated by the process of production from the reservoir. Accordingly, methods of the present disclosure are valuable predictors of ultimate production in a source reservoir. For purposes of clarity, “production” comprises the process of drilling a well to penetrate a reservoir for producing fluids (e.g., gas and/or liquids) from one or more subterranean formations. “Production” may also include operations, generally referred to as stimulation, to improve the productivity index of a well and/or the surrounding formation. For example, stimulation operations (e.g., acidizing) may be used to remove damage zones (e.g., skin) proximate to the wellbore. In many instances, in particular in tight formations such as shale, the reservoir formation is fractured utilizing high pressure applied from the well. The induced fractures may be propped open, for example using a proppant, to maintain the increased permeability provided by the fractures.

The generation of methane, for example, from higher hydrocarbons is an energetically favored reaction at low temperatures. For example, ΔG for butane decomposition to methane, ethane and carbon in unspecified form is −15.9 kcal/mol at 25° C., which indicates the possibility of a spontaneous reaction occurring. The reaction for the decomposition of butane is given in Formula (1).

C₄H₁₀→CH₄+C₂H₆+—C— (Formula 1)

The reaction is exceedingly slow in the absence of a catalyst but much faster in the presence of a catalyst. A rock sample that fails to generate gas in a laboratory setting is therefore expected to be obtained from a petroleum habitat, rather than a gas habitat, since it is unlikely to contain transition metal catalysts to facilitate the decomposition of higher hydrocarbons into gas. Hence, methods of the present disclosure may be used for identifying drilling sites likely to produce oil or to produce gas, depending on the outcome of the assay for catalytically-generated gas, such as methane, in a rock sample obtained from the drilling site.

Carbonaceous sedimentary rocks include, for example, shales containing kerogens (siliceous and carbonate) often referred to as ‘source rocks’, coals, tar sands, and reservoir rocks containing residual oil. Non-carbonaceous sedimentary rocks include, for example, sandstones and carbonate rocks, which contain inorganic carbon. Both carbonaceous sedimentary rocks and non-carbonaceous sedimentary rocks may contain transition metals, which may be LVTM, ZVTM or a combination thereof. Carbonaceous sedimentary rocks containing transition metals show the ability to catalytically generate gas, whereas non-carbonaceous sedimentary rocks do not have the ability to catalytically generate gas, as carbonates are unaffected by transition metal catalysts. Therefore, identification of a rock sample as being able to catalytically generate gas in a laboratory environment may be correlated with the ability to generate gas in a geological formation from which the rock sample is derived. In some embodiments, the quantity of methane generated correlates with an intrinsic catalytic activity of the rock sample.

According to one or more aspects of the present disclosure, the intrinsic catalytic activity of a rock sample may be projected on to a source reservoir from which the rock sample originated and the amount of catalytically-generated gas capable of being produced by the source reservoir predicted. In some embodiments, the intrinsic catalytic activity may be projected on to a genetically-similar source reservoir and the amount of catalytically-generated methane capable of being produced by the genetically-similar reservoir predicted.

According to one or more aspects of the present disclosure a method for assaying a rock sample for the presence of catalytically-generated gas, for example methane, comprises preparing a rock sample comprising a carbonaceous material; sealing the prepared rock sample in a container; maintaining the prepared rock sample in the container for a period of time; and assaying for a quantity of gas in the container after the period of time has elapsed. The rock sample may comprise a low-valent transition metal. Preparing the rock sample may comprise grinding. According to one or more aspects of the present disclosure, the container comprises a static atmosphere (e.g., non-flowing) when the container is sealed. In at least one embodiment, the rock sample is maintained at a temperature less than about 100° C. while maintained in the container. In some embodiments, the static atmosphere is an inert atmosphere such as, for example, argon, nitrogen or helium. In other embodiments, the static atmosphere is at least partially air.

In some embodiments, sealing the rock sample in the container takes place with a septum. Use of a septum advantageously allows for the container to be periodically sampled for the qualitative or quantitative presence of methane gas without having to unseal the container. For example, in various embodiments, the septum may be pierced with a syringe needle to sample for the presence of gas inside the container.

In various embodiments, the preparation of the rock sample takes place before sealing the rock sample in the container. For example, preparing the rock sample may include converting the as-obtained rock sample into smaller pieces. Such conversion may be accomplished by actions such as, for example, grinding, milling, pulverizing, treating by ultrasound, and blending. Without being bound by theory or mechanism, it is believed that preparing the rock sample by grinding or otherwise breaking the rock sample into smaller pieces increases surface area and brings pools of carbonaceous material into contact with transition metals, which then catalyze the conversion of the carbonaceous material into gas. Once the carbonaceous material in contact with the catalytic transition metals in the rock source is converted to gas, gas generation ceases. The proposed mechanism is consistent with a single gas generation event, which is observed experimentally.

In natural geological settings, transition metals are likely isolated from surrounding carbonaceous materials. Since mass transport is diffusion controlled and occurs slowly over geologic time, only slow conversion of petroleum to gas is observed geologically. As noted above, processing of the rock sample by grinding or other physical means brings the carbonaceous material into contact with surrounding catalytic sites and dramatically speeds up catalytic gas generation under experimental conditions. Likewise, any physical process that brings surrounding carbonaceous material into contact with the transition metals in a rock formation may stimulate catalytic gas production in the formation. Geologically, this process can occur through faulting, uplifting, natural fracturing, and other physical processes that stress rocks. For petroleum exploration, drilling, fracturing and gas flow with production also have the potential for stimulating gas generation by bringing the transition metals into contact with surrounding carbonaceous material.

In various embodiments described herein, the rock samples are ground. Such grinding can be accomplished, for instance, by hand with a mortar and pestle or through mechanical milling. In a non-limiting embodiment, the rock sample can be milled with the sample placed in a closed cylinder containing a brass ball and shaking the cylinder with a mechanical ‘paint shaker’ for a period of time such as, for example, 15 minutes. Mechanical rock crushing in brass prevents sample contamination by transition metals in steel cylinders and balls. There can be considerable variability in the mesh size and surface area of the particles after grinding. For example, in various embodiments, the rock samples are ground to be at least about 60 mesh in size. In some embodiments, the ground sample is sieved to include or exclude particles of a particular size or range of sizes.

In some embodiments, preparation of the quantity of rock sample is conducted under an inert atmosphere. Processing under inert atmosphere is conducted to avoid any potential oxidation of transition metals into a non-catalytic state. Inert atmospheres include, for example, helium, nitrogen, and argon atmospheres and combinations thereof.

In some embodiments of the present disclosure, preparation of the quantity of rock sample is conducted in air. Applicant has found that preparation of the rock sample in air does not destroy the catalytic activity of transition metals present in the rock sample, as conventionally believed in the art. As demonstrated by example herein, processing under inert atmosphere is not required for catalytic activity, but it may influence the observed intrinsic catalytic activity in certain cases. For example, oxidation may lead to an observed intrinsic catalytic activity that is below the true value for the rock sample in certain instances. However, the observed catalytic activity may be correlated with the intrinsic catalytic activity, even when the observed catalytic activity is impacted by oxidation. By processing and assaying the rock samples under inert conditions, the intrinsic catalytic activity of the rock sample may be directly determined.

There is a wide range of the quantity of rock sample used in the examples described herein. The amount of rock sample used is between about 0.1 g and about 20 g in some examples, between about 0.5 g and about 10 g in other examples, or between about 0.5 g and about 5 g in still other examples.

In some embodiments, the rock sample may be heated before or after being prepared for the gas assays of the present disclosure. In some embodiments, the heat treatment removes any pre-existing, non-catalytically generated hydrocarbons, including methane, from the rock sample that could be mistaken for catalytically-generated gas in the assays described herein.

In some embodiments, the rock sample is kept at room temperature while being maintained in the container for the period of time. In some embodiments, the rock sample is heated at a temperature between room temperature and about 400° C. while being maintained in the container for the period of time. In some embodiments, the rock sample is heated at a temperature between room temperature and about 100° C. while being maintained in the container for the period of time.

Generally, in conventional rock assays for determining oil-to-gas conversion activity, the rock samples are heated in the presence of a flowing stimulation gas at a temperature of about 150° C. or higher. Such methods utilizing flowing stimulation gas are described in commonly-assigned United States Published Patent Application No. 2008-0115935. Stimulation gases typically include inert, non-oxidizing gases that are substantially oxygen-free in order to maintain any ZVTM or LVTM present in the rock samples in a catalytically-active state. Stimulation gases include, for example, He, Ar and N₂. Under such conditions, the rock samples tend to generate gas episodically in a chaotic fashion. Again without being bound by theory or mechanism, Applicant believes that the episodic gas generation is a mass transport phenomenon wherein the carbonaceous material is transported by the flowing stimulation gas to the catalytically-active ZVTM or LVTM sites. The kinetics of the conversion reaction is chaotic.

The single gas generation event of the described rock assays is typically complete within several hours of sealing the prepared rock sample in the container. In some embodiments, the sample is maintained in the container for a period of time between about 1 minute and about 12 hours before assaying for the quantity of gas (e.g., methane). In other embodiments, the sample is maintained in the container for a period of time between about 1 minute and about 1 hour before assaying for the quantity of gas.

In some embodiments, the container for the rock sample contains an inert atmosphere after being sealed. Inert atmospheres include at least one gas such as, for example, helium, nitrogen and argon. In some embodiments, the inert atmosphere is static. In other embodiments, the container for the rock sample contains an atmosphere that is at least partially air after being sealed. In some embodiments, the atmosphere containing air is static.

In contrast to conventional rock assays, methods of the present disclosure may be conducted in a static environment (e.g., without flowing stimulation gas), which advantageously changes the kinetics of gas production. Although some embodiments of the present disclosure utilize an inert atmosphere in analyzing the rock samples, there is no general requirement to do so. Again without being bound by theory or mechanism, Applicant believes that the flowing stimulation gas of conventional rock assays delivers larger quantities of oxygen to ZVTM or LVTM catalytic sites, resulting in their oxidation and poisoning. In contrast, the static environment of the present rock assays does not deliver sufficient quantities of oxygen to the ZVTM or LVTM catalytic sites to result in poisoning, even when an inert gas is not used when sealing the container in which the rock sample is contained.

As a further distinction over conventional rock assays, catalytic gas generation according to one or more aspects of the assays of the present disclosure occurs in a single episode over the course of a few minutes or hours, as opposed to the chaotic, episodic gas generation in the presence of a flowing stimulation gas. Accordingly, the rock assays of the present disclosure may have significant advantages in being simpler to conduct and in providing simpler data output.

Typically, the rock sample is obtained from a reservoir of interest, such that data collected for the rock sample is representative of the reservoir and genetically-similar reservoirs. In some embodiments, the quantity of gas (e.g., methane) produced from the rock sample correlates with an intrinsic catalytic activity of the rock sample. In some embodiments of the present disclosure, the methods further include projecting the intrinsic catalytic activity on to a source reservoir from which the rock sample originated and predicting an amount of catalytically-generated gas capable of being produced by the source reservoir. In further embodiments, the methods of the present disclosure further include projecting the intrinsic catalytic activity on to a genetically-similar source reservoir and predicting an amount of catalytically-generated gas (e.g., methane) capable of being produced by the genetically-similar source reservoir.

When assaying a rock sample as described above, qualitative analysis of any catalytically-generated gas (e.g., methane) may be sufficient to make predictive assessments as to the content (i.e., primarily oil or primarily gas) of the reservoir from which the rock sample was extracted (source reservoir) or of any other reservoir that is genetically similar to the source reservoir. In some or other embodiments, however, a quantitative analysis of catalytically-generated gas (e.g., methane) provides greater insight into the content of such a source reservoir. For example, rock samples generating more gas (e.g., methane) per weight unit of rock may have more capacity for yielding gas (e.g., methane) in a source reservoir. Further, the amount of gas (e.g., methane) generated in the present rock assay may be predictive of the sustainability of production from a particular well.

In some embodiments, a separation of methane from other gaseous hydrocarbons emitted from the rock sample may be performed. Such gaseous hydrocarbons may also be generated from the catalytic decomposition of higher hydrocarbons. For example, other C2-C5 hydrocarbons may be concurrently produced with methane.

In some embodiments, a mass quantity of catalytically-generated methane is determined after separation. One or more separation steps may be performed on the catalytically-generated gas. Such separation steps can be used to separate catalytically-generated methane from other gaseous hydrocarbon species. In some embodiments, the separating step includes use of a cold trap (e.g., a liquid nitrogen trap) that condenses all other hydrocarbons on the cold trap, but does not condense methane. In other embodiments, a chromatographic separation is employed. Chromatographic separation may include a gas chromatograph separation, wherein any suitable stationary phase may be used to separate methane from other gaseous hydrocarbon species.

In some embodiments, detecting the presence of catalytically-generated gas includes use of a gas chromatograph. The gas chromatograph can have a number of detectors such as, for example, a flame ionization detector (FID), a mass-selective detector, a spectroscopic detector, an electron capture detector, a thermal conductivity detector, a residual gas analyzer, and combinations thereof. In some embodiments, the optional step of separating is coupled with detection and analysis of the catalytically-generated gas.

In some embodiments, oil and/or gas predictions can be made on a reservoir other than the source reservoir if the two reservoirs share a common depositional environment and thus can be expected to be similar in overall composition and transition metal content (i.e., genetically-similar reservoirs). Methods and assays described herein can potentially predict the presence of oil and/or gas in an un-drilled reservoir based on analysis of rock samples taken from drilled genetically similar reservoirs distal from the un-drilled reservoir. In other embodiments, stratigraphic units can be mapped for catalytic activity by assaying representative rock samples covering the various depositional environments throughout the stratigraphic units. From the paleocatalytic activities at depth and residence times, habitat maps can be constructed showing where in these units oil will convert to gas and where it should not, thus where in the basin the probability for oil is high (oil habitats) and where it is low (gas habitats). Habitat maps could be particularly useful in mapping sedimentary rocks that are particularly rich in transition metals such as, for example, the outer-neritic shales.

Some embodiments of the present disclosure enable prediction of the distribution of oil and gas in various reservoirs within a stratigraphic rock unit based on an oil-gas habitat map. Some embodiments of the present disclosure enable prediction of the distribution of oil and gas in various reservoirs in a stratigraphic rock unit proximal to a stratigraphic source rock unit within which oil and gas is generated and expelled into reservoirs within the proximal rock unit based on the oil-gas habitat map of the source rock unit. Some embodiments of the present disclosure enable a prediction of the conversion of oil to gas within a conduit rock along an oil migration pathway

In economic terms, if a reservoir is sufficiently removed from natural gas markets, then the economic incentives for drilling in an oil habitat greatly outweigh those for drilling in a gas habitat. Methods of the present disclosure may advantageously permit such determinations to be made inexpensively with a relatively high level of accuracy, helping to avoid significant and costly exploration processes in order to ascertain the reservoir's content.

While the embodiments described herein have focused primarily on catalytic activity afforded by transition metals, the possibility that other low-valent or zero-valent metals catalyze conversion of oil to gas should not be excluded and lie within the spirit and scope of the present disclosure. For example, it is possible that low- or zero-valent rare earth metals, catalyze oil-to-gas conversion in certain embodiments of the methods described herein.

### Experimental Examples

The following examples are provided to demonstrate particular embodiments of the present disclosure. It should be appreciated by those of ordinary skill in the art that the methods disclosed in the examples merely represent illustrative embodiments that should not be considered limiting. Those of ordinary skill in the art should, in light of the present disclosure, appreciate that many changes can be made in the specific embodiments described and still obtain a like or similar result without departing from the spirit and scope of the present disclosure.

### Example 1

### General Assay Procedure for a Carbonaceous Rock Sample Using Barnett Shale

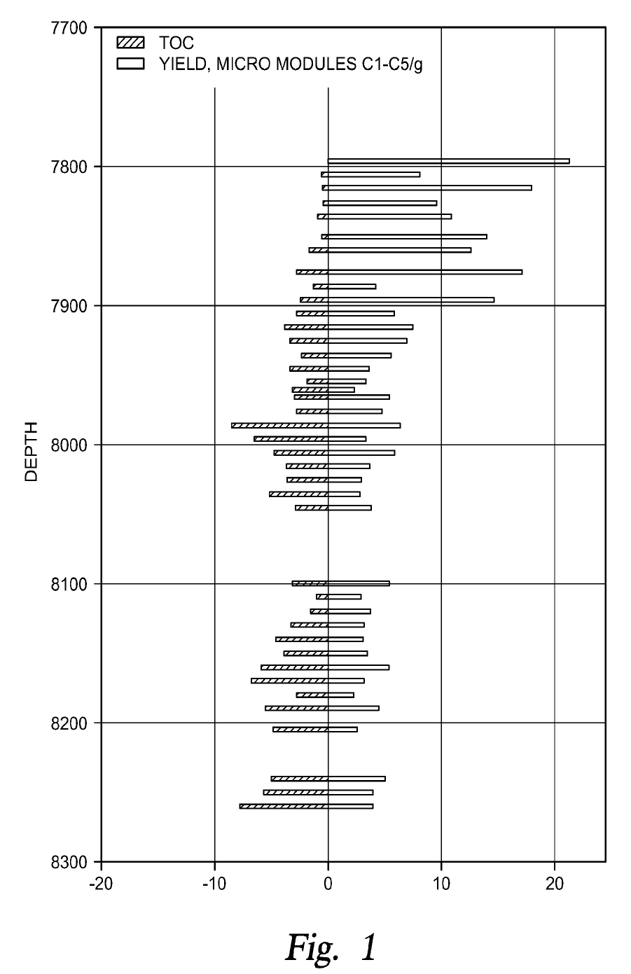

About 4 g of a Barnett shale from Montague county, TX (Jenny #1 well) (cuttings, 7825 ft) was ground to 60 mesh particles with mortar and pestle in Ar. About 2 g of 60 mesh shale was weighed (2.21 g) and placed in a 5 ml glass vial and sealed with a screw-cap vial with a silicone/Teflon septum. The weighing and sealing operations were conducted under Ar. The vial was then placed in an oven at 100° C. for one hour. A 250 μl aliquot of gas was taken from the vial and passed directly into the injector port of a gas chromatograph (GC) unit through a 6-way valve. GC analysis (commercial columns) gave base-line separation of all hydrocarbons between C1 and C5. The yield of hydrocarbons was similar to that obtained via assay conditions described in U.S. Pat. No. 7,153,688, which is incorporated herein by reference. FIG. 1 is an illustrative log of total organic carbon and C1-C5 hydrocarbon in Barnett Shale by depth as analyzed by a method according to one or more aspects of the present disclosure. The gas yield was 4.5 μg C1-C5/g (22 nmol C1-C5/g).

### Example 2

### Assay of Mahogany Shale Under Anoxic Conditions

A sample of Mahogany shale was analyzed for catalytic gas generation at various temperatures and for various times in this Example. The shale sample was taken from the Tertiary Green River Formation, Mahogany Zone, Section 13, T10S, R24E, Uintah County, Utah (Mahogany). About 1 gm of the sample was ground to 60 mesh in an argon bag and placed in a 5 ml glass vial, which was then sealed with a rubber septum. The vials were heated at various temperatures for various periods of time and then analyzed by gas chromatography as above. Table 1 shows the gas yield in μg C1-C5/g rock.

TABLE 1
Anoxic Gas Generation in Closed Reactor, Mahogany Shale

| Temperature (°C.) | Time (min) | Yield (μg gas/g) |
|---|---|---|
| 35 | 0 | 0.52 |
| 35 | 60 | 1.0 |
| 35 | 120 | 1.6 |
| 100 | 60 | 29 |
| 100 | 120 | 32 |

### Example 3

### Assay of Floyd Shale Under Anoxic Conditions

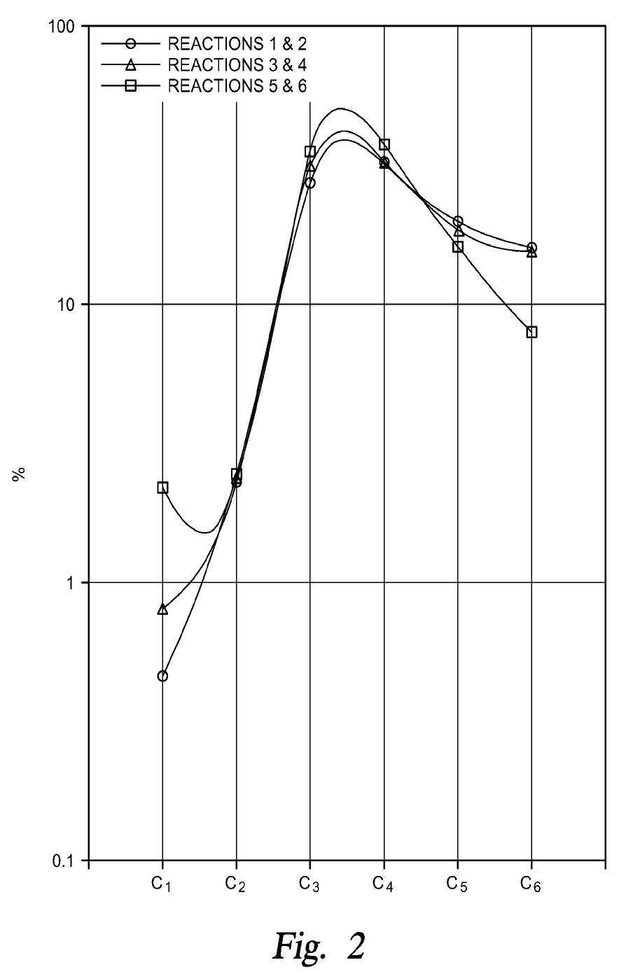

A sample of Floyd shale was analyzed for catalytic gas generation for various times and at various temperatures in this Example. A Mississippian Floyd Shale sample from the Black Warrior Basin in Clay County, Mississippi was obtained. Processing and analysis was conducted as in Example 2. Table 2 shows the gas yield in μg C1-C5/g rock. Table 3 shows the distribution of C1-C5 hydrocarbons catalytically-generated from Floyd Shale as a function of time at 50° C. FIG. 2 is an illustrative distribution of total C1-C5 hydrocarbons catalytically-generated from Floyd Shale at 50° C. under static He conditions.

TABLE 2
Anoxic Gas Generation in Closed Reactor, Floyd Shale

| Temperature (°C.) | Time (min) | Yield (μg gas/g) |
|---|---|---|
| 35 | 0 | 3.6 |
| 35 | 60 | 3.6 |
| 35 | 120 | 2.7 |
| 35 | 180 | 3.1 |
| 100 | 60 | 16 |
| 100 | 120 | 22 |
| 100 | 180 | 23 |

TABLE 3
C1-C5 Distribution in Floyd Shale at
50° C. as a Function of Time (mol %)

| | 1^{st} | 2^{nd} | 3^{rd} | 4^{th} | Next | Next |
|---|---|---|---|---|---|---|
| | Hour | Hour | Hour | Hour | 19 Hr | 19 Hr |
| Methane | 0.24 | 0.88 | 1.34 | 0.58 | 1.21 | 3.28 |
| Ethane | 3.11 | 2.77 | 2.66 | 2.65 | 2.82 | 1.75 |
| Propane | 35.87 | 35.04 | 35.46 | 39.17 | 41.74 | 36.75 |
| i-Butane | 11.25 | 10.39 | 10.25 | 9.54 | 13.05 | 13.67 |
| n-Butane | 27.05 | 27.06 | 27.81 | 27.46 | 25.29 | 26.37 |
| i-Pentane | 11.27 | 11.18 | 10.30 | 9.20 | 8.33 | 9.50 |
| n-Pentane | 11.21 | 12.67 | 12.19 | 11.41 | 7.55 | 8.67 |
| Cumulative | 0.204 | 0.263 | 0.304 | 0.358 | 0.422 | 0.445 |
| C1-C5 | | | | | | |
| (μmol/g) | | | | | | |

### Example 4

### Assay of Floyd Shale Under Oxic Conditions

A sample of Floyd shale was analyzed for catalytic gas generation as in Example 2, except the sample was processed under oxic conditions. A Mississippian Floyd Shale sample from the Black Warrior Basin in Clay County, Mississippi was obtained. The shale sample was ground to 60 mesh in air, sealed in 5 ml glass reactors in air and treated and analyzed as described in Example 2. Except for the products obtained at room temperature, gas generation under oxic conditions in closed containers showed no significant differences from the same reactions conducted under anoxic conditions. Table 4 shows the gas yield in μg C1-C5/g rock.

TABLE 4
Gas Generation Under Oxic Conditions in
Closed Reactor, Floyd Shale

| Temperature (°C.) | Time (min) | Yield (μg gas/g) |
|---|---|---|
| 35 | 0 | 1.0 |
| 35 | 60 | 2.0 |
| 100 | 60 | 15 |
| 100 | 120 | 19 |
| 100 | 180 | 31 |

### Example 5

### Assay of Floyd Shale from a Different Stratigraphic Region Under Anoxic Conditions

A sample of Floyd shale from a different stratigraphic region was analyzed for catalytic gas generation as in Example 2. The sample was processed under anoxic conditions. Table 5 shows the gas yield in μg C1-C5/g rock.

TABLE 5
Anoxic Gas Generation in Closed Reactor,
Second Sample of Floyd Shale

| Temperature (°C.) | Time (min) | Yield (μg gas/g) |
|---|---|---|
| 35 | 0 | 45 |
| 100 | 60 | 204 |
| 100 | 120 | 191 |
| 100 | 180 | 223 |
| 100 | 1400 | 127 |

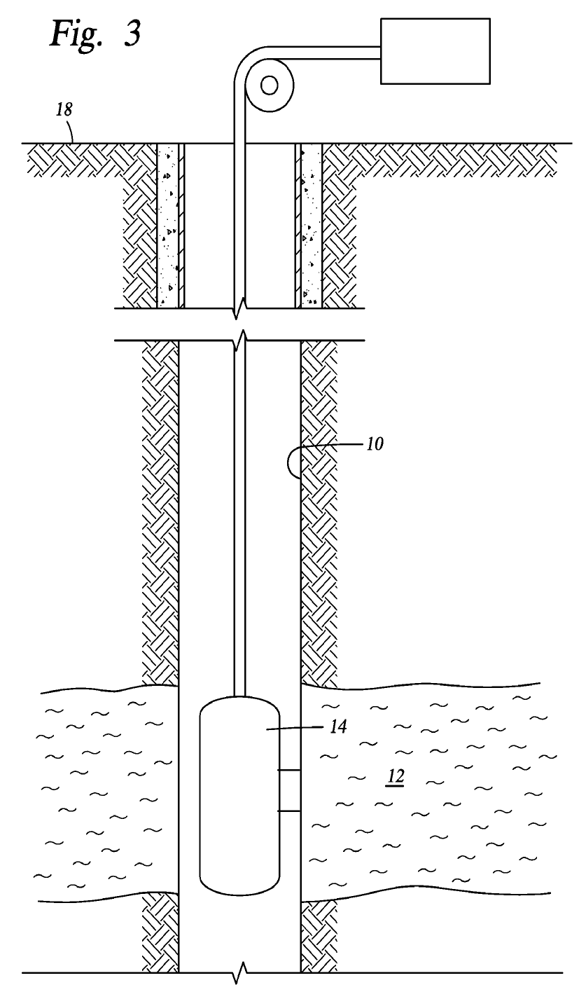

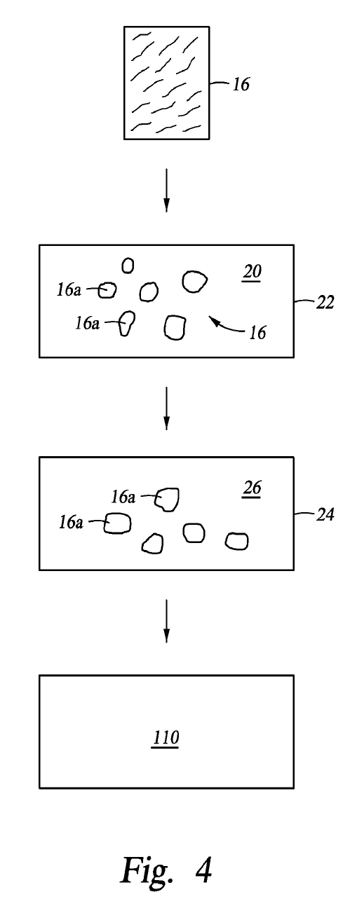

FIG. 3 is a schematic illustration depicting a well 10 (e.g., wellbore) intersecting a target reservoir 12. Target reservoir 12 comprises sedimentary rock having a carbonaceous material. A wellbore tool 14 is lowered into well 10 to target reservoir 12 to obtain a rock sample 16 (FIG. 4). Tool 14 may be deployed via wireline or a tubular. According to one or more aspects of the present disclosure, tool 14 may be a coring tool, for example a side-wall coring device as depicted in FIG. 3. Tool 14 may be a formation evaluation tool wherein in all or part of one or more of the assays disclosed herein may be performed. For example, a rock sample may be obtained and the assayed performed while tool 14 is disposed downhole in wellbore 10. According to one or more aspects of the present disclosure, the rock sample may be obtained downhole as depicted in FIG. 3 and raised to the surface 18 wherein the assay may be performed at the well site or at a location (e.g., laboratory) remote from the well site.

FIG. 4 is a schematic diagram a method according to one or more aspects of the present disclosure. In this embodiment, rock sample 16 is initially provided in step 100 in a single portion of material. In this embodiment, the obtained rock sample 16 is broken into a plurality of smaller pieces 16a in a step 104. Rock sample 16 is broken into pieces (e.g., prepared) in an atmosphere 20. Atmosphere 20 may comprise air or may be an inert atmosphere. Rock sample 16 may be broken into pieces (step 104) in a closed environment such as a container 22. Container 22 may be located downhole in tool 14 of FIG. 3 for example. In step 108, rock sample 16 is sealed in a container 24 having an atmosphere 26. Container 22 and container 24 may be the same or different containers. Again, the rock sample may be sealed in a container that is disposed in well 10, for example in tool 14. In some embodiments, atmosphere 26 of sealed container 24 comprises air. In some embodiments, atmosphere 26 of sealed container 24 is inert. In the depicted embodiment, atmosphere 26 of sealed container 24 is static. Rock sample 16 may be maintained in sealed container 24 of a predetermined period of time. The temperature in sealed container 24 may be maintained at a substantially constant temperature, for example a room or ambient temperature, or it may be heated. Sealed container 24 is assayed (step 110) for a quantity of catalytically generated gas.

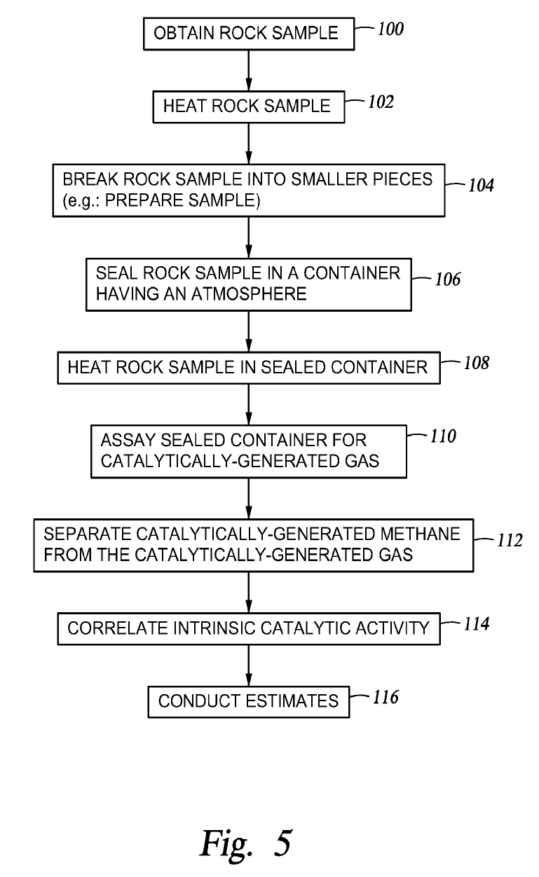

FIG. 5 is a schematic diagram of another embodiment of an assay according to one or more aspects of the present disclosure. In step 100 a rock sample is obtained, for example from a wellbore as depicted in FIG. 3 or by other means. In a step 102 of this embodiment, the rock sample is heated for example to remove existing gas from the rock sample. In step 104 of this embodiment, the rock sample is broken into smaller pieces. In step 106, the rock sample is sealed in a container having an atmosphere. The atmosphere in this embodiment is static. In step 108, the sealed container, in particular the atmosphere and contained rock sample, is heated. In step 110 the sealed container is assayed for catalytically-generated gas, for example for the quantity of catalytically-generated gas. In step 112, methane (e.g., catalytically-generated methane) may be separated from the total catalytically-generated gas. In step 114, the quantity of COG assayed may be correlated with an intrinsic catalytic activity of the rock sample. In step 116, the assay results (step 110) and/or intrinsic catalytic activity (step 112) may be utilized to estimate (e.g., project) the catalytic activity of a source rock (e.g., target formation) and/or catalytically-generated gas that may be produced from a quantity of similar rock for example.

A method according to one or more aspects of the present disclosure comprises sealing a carbonaceous rock sample in a container having an atmosphere; and assaying for a quantity of catalytically generated gas in the sealed container. The method may comprise estimating a quantity of catalytically-generated gas that may be produced from a subterranean source reservoir.

The carbonaceous rock sample may comprise a plurality of pieces of carbonaceous rock. The method may comprise breaking the carbonaceous rock sample into smaller pieces prior to sealing in the container. The breaking of the rock sample into smaller pieces may be conducted under and an inert atmosphere. The inert atmosphere comprises a gas selected from the group of helium, nitrogen and argon. The breaking may be conducted in an air atmosphere.

The method may comprise heating the carbonaceous rock sample prior to sealing. The carbonaceous rock sample may be heated prior to breaking. The rock sample may also be heated when it is disposed (e.g., contained) in the sealed container.

The atmosphere of the sealed container may be inert or comprise air. The atmosphere may be static. The sealed container, while containing the rock sample, may be heated or maintained at an ambient temperature.

The method may comprise generating catalytic-generated gas from the carbonaceous rock sample in the sealed container. The catalytic-generated gas may be generated in response to a catalytic reaction between a carbonaceous material in the carbonaceous rock sample and a low-valent transition metal present in the carbonaceous rock sample.

The method may comprise correlating the quantity of catalytically-generated gas assayed with an intrinsic catalytic activity of the carbonaceous rock sample. The method may comprise correlating the quantity of catalytically-generated gas assayed with an intrinsic catalytic activity of the carbonaceous rock sample; and projecting the intrinsic catalytic activity on to a source reservoir

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure. The scope of the invention should be determined only by the language of the claims that follow. The term “comprising” within the claims is intended to mean “including at least” such that the recited listing of elements in a claim are an open group. The terms “a,” “an” and other singular terms are intended to include the plural forms thereof unless specifically excluded.

What is claimed is:

1. A method for assaying a rock sample, comprising:
sealing a carbonaceous rock sample in a container having an atmosphere; and
assaying for a quantity of catalytically generated gas in the sealed container.

2. The method of claim 1, wherein the carbonaceous rock sample comprises a plurality of pieces of carbonaceous rock.

3. The method of claim 1, comprising breaking the carbonaceous rock sample into smaller pieces prior to sealing.

4. The method of claim 3, wherein breaking is conducted under an inert atmosphere.

5. The method of claim 4, wherein the inert atmosphere comprises a gas selected from the group of helium, nitrogen and argon.

6. The method of claim 3, wherein breaking is conducted in an air atmosphere.

7. The method of claim 1, further comprising heating the carbonaceous rock sample prior to sealing.

8. The method of claim 3, further comprising heating the carbonaceous rock sample prior to breaking.

9. The method of claim 1, wherein the atmosphere is inert.

10. The method of claim 1, wherein the atmosphere comprises air.

11. The method of claim 1, wherein the atmosphere is static.

12. The method of claim 1, further comprising heating the sealed container.

13. The method of claim 1, further comprising:
heating the carbonaceous rock sample prior to sealing in the container; and
heating the sealed container.

14. The method of claim 1, further comprising maintaining sealed container at a room temperature.

15. The method of claim 1, further comprising heating the sealed container to a temperature of about 400° C.

16. The method of claim 1, further comprising heating the sealed container to a temperature of about 100° C.

17. The method of claim 1, comprising maintaining the carbonaceous rock sample in the sealed container for a period of time prior to assaying.

18. The method of claim 1, comprising maintaining the carbonaceous rock sample in the sealed container for a period of time prior to assaying, wherein the period of time comprises the range of 1 minute to about 12 hours.

19. The method of claim 1, further comprising generating catalytic-generated gas from the carbonaceous rock sample in the sealed container.

20. The method of claim 1, further comprising generating catalytic-generated gas from the carbonaceous rock sample in the sealed container in response to a catalytic reaction between a carbonaceous material in the carbonaceous rock sample and a low-valent transition metal present in the carbonaceous rock sample.

21. The method of claim 1, further comprising:
generating catalytic-generated gas from the carbonaceous rock sample in the sealed container; and
separating methane from the catalytically generated gas.

22. The method of claim 1, further comprising correlating the quantity of catalytically-generated gas assayed with an intrinsic catalytic activity of the carbonaceous rock sample.

23. The method of claim 1, further comprising:
correlating the quantity of catalytically-generated gas assayed with an intrinsic catalytic activity of the carbonaceous rock sample; and
projecting the intrinsic catalytic activity on to a source reservoir.

24. The method of claim 1, further comprising estimating a quantity of catalytically-generated gas that may be produced from a subterranean source reservoir.
